(19) United States
(12) Patent Application Publication
(10) Pub. No.: US 2002/0183300 A1
(43) Pub. Date: Dec. 5, 2002

(54) Zinc ionophores as anti-stress agents

(76) Inventor: Henry Fliss, Ottawa (CA)

Correspondence Address: SCULLY, SCOTT, MURPHY & PRESSER, 400 Garden City Plaza, Garden City, NY, 11530, US

(21) Appl. No.: 10/116,535

(22) Filed: Apr. 4, 2002

Related U.S. Application Data
(60) Provisional application No. 60/281,490, filed on Apr. 4, 2001.

(51) Int. Cl. A61K 31/555; A61K 31/325; A61K 31/315

(52) U.S. Cl. 514/184; 514/186; 514/478; 514/494

(57) ABSTRACT

The present invention provides methods comprising one or more zinc ionophores for treating or reversing the effects of stress, including surgical stress in patients in need thereof.

### CROSS-REFERENCE TO RELATED APPLICATIONS

This application claims the benefit of U.S. Provisional Application Serial No. 60/281,490, filed Apr. 4, 2001.

### FIELD OF THE INVENTION

The present invention provides methods for modulating the effects of stress, including surgical stress in patients in need thereof by the administration of one or more zinc ionophores. The present invention also provides methods of regulating gene expression by modulating the activity of transcription factors in various organ systems of mammals, including the brain, by administering to a patient in need thereof a pharmaceutically effective amount of at least one zinc ionophore.

### BACKGROUND OF THE INVENTION

Different forms of stress, such as surgical stress, are known to alter brain physiology and may lead to brain injury (McEwen B. S. (2000) Brain Res. 886:172-189; DeKeyser F. G. et al. (2000) Neuroimmunomodulation 7:182-188; Revilla V. et al. (1999) Brain Res. Bull 49:413-418; Truollos E. et al. (1997) Clin. Pharmacol. Ther. 62:74-81). The physiological effects of stress can involve complex hormonal changes and may lead to alterations in the activity of a number of transcription factors (Udelsman R. et al. (1994) Curr. Probl. Surg. 31:653-720). In turn, the transcription factors may modulate the neuronal response to the stress (Pennypacker K. R. et al. (2000) Acta Neurobiol. Exp. (Warsz) 60:515-530; Pennypacker K. (1998) Int. Rev. Neurobiol 42:169-197; Pennypacker K. (1997) Histol Histopathol 12:1125-1133). The inventor has shown previously that zinc ionophores can modulate the activity of a number of transcription factors in endothelial cells in vitro, as well as in rat brain in vivo (U.S. Ser. No. 09/759,091 entitled “Zinc Ionophores as Anti-Apoptotic Agents”, filed Jan. 12, 2001, docket 13595Z, incorporated herein by reference).

Zinc plays a critical role in cellular biology, and is involved in virtually every important cellular process such as transcription, translation, ion transport, and others (O'Halloran, T. V. (1993) Science 261:715-725; Cousins, R. J. (1994) Annu.Rev.Nutr. 14:449-469; Harrison, N. L. et al. (1994) Neuropharmacology 33:935-952; Berg, J. M. et al. (1996) Science 271:1081-1085). The involvement of cellular zinc in apoptosis has been recognized for close to twenty years (Sunderman, F. W.,Jr. (1995) Ann.Clin.Lab.Sci. 25:134-142; Fraker, P. J. et al. (1997) Proc.Soc.Exp.Biol.Med. 215:229-236.). However, the full nature of this involvement is not fully understood. Apoptosis is a form of programmed cell death normally activated under physiological conditions, such as involution in tissue remodelling during morphogenesis, and several immunological processes. The apoptotic process is characterized by cell shrinkage, chromatin condensation, and intemucleosomal degradation of the cell's DNA (Verhaegen et al. (1995) Biochem. Pharmacol. 50(7):1021-1029).

Numerous in vitro studies have been done recently in an attempt to elucidate the role of intracellular zinc. Although some studies have suggested that zinc may actually induce apoptosis (Xu, J. et al. (1996) Am.J.Physiol. 270:G60-G70; Kim, Y. H. et al., (1999) Neuroscience 89:175-182), most have concluded that increasing the intracellular concentrations of zinc blocks apoptosis (Sunderman, F. W.,Jr. (1995) Ann.Clin.Lab.Sci. 25:134-142; Adebodun, F. et al. (1995) J.Cell.Physiol. 163:80-86; Zalewski, P. D., et al. (1993) Biochem.J. 296:403-408), and that decreasing the zinc concentration promotes apoptosis (Jiang, S., et al. (1995) Lab.Invest. 73:111-117; Treves, S., et al. (1994) Exp.Cell Res. 211:339-343; Ahn, Y. H., et al. (1998) Exp.Neurol. 154:47-56). The manner in which increased intracellular zinc affords protection against apoptosis is not clear. (Truong-Tran, A. Q. et al., (2000) J. Nutr. 130:1459S-1466S) One theory proposes that zinc inactivates the intracellular endonuclease(s) responsible for apoptotic DNA fragmentation (Shiokawa, D., et al. (1994) Eur.J.Biochem. 226:23-30; Yao, M. et al., (1996) J.Mol.Cell.Cardiol. 28:95-101). Other recent studies have suggested that zinc can inhibit caspases (Jiang, S., et al. (1997) Cell Death Differ. 4:39-50; Perry, D. K., et al. (1997) J.Biol.Chem. 272:18530-18533; Maret, W., et al. (1999) Proc.Natl.Acad.Sci.USA 96:1936-1940), or block the activation of caspases (Aiuchi, T., et al. (1998) J.Biochem. 124:300-303). However, in view of the large number of intracellular roles played by zinc, it seems likely that its anti-apoptotic mechanisms may be more complex, possibly involving gene expression and cellular signalling pathways. In a recent study, animals subjected to stress have been shown to have altered gene expression profiles (Meshorer et al. (2002) Science 295:508-512). In fact, studies support a role for zinc transients in intracellular signalling and gene expression (O'Halloran, T. V. (1993) Science 261:715-725; Berg, J. M., et al., (1996) Science 271:1081-1085).

In contrast to the large number of in vitro studies, very few studies have attempted to examine the protective effects of zinc in vivo. It is important to note that most of the studies that have explored this possibility have focused on the pretreatment of tissues with zinc prior to injury. Using this approach, a number of studies have demonstrated that pretreatment of animals with zinc at least 24 hours prior to injury provided some measure of protection against apoptosis (Thomas, D. J. et al., (1991) Toxicology 68:327-337; Matsushita, K., et al., (1996) Brain Res. 743:362-365; Klosterhalfen, B., et al., (1997) Shock 7:254-262), presumably as a result of the well established ability of zinc to boost the immune system (Cunningham-Rundles, S., et al., (1990) Ann.N.Y.Acad.Sci. 587:113-122). Also, one study showed that several days of zinc dietary supplementation concomitant with i.p. injection of carbon tetrachloride protected against liver apoptosis (Cabre, M. et al. (1999) J. Hepatol. 31:228-234). However, no studies have demonstrated the effect of zinc ionophores on the activity of transcription factors in the brain, following surgical stress. Moreover no studies have demonstrated that zinc ionophores can influence the cascade of physiological events triggered by stress and thereby treat and/or prevent the deleterious effects of stress.

### SUMMARY OF THE INVENTION

The present invention is directed to the use of zinc ionophores including, but not limited to zinc-pyrithione (ZnP) and zinc-diethyldithiocarbamate (ZNDDC) to reverse the effects of stress and especially surgical stress in mammals. The present invention is also directed to the use of zinc ionophores including, but not limited to zinc-pyrithione (ZnP) and zinc-diethyldithiocarbamate (ZNDDC) to modulate the effects of surgical stress on the activity of transcription factors, including but not limited to NF-kB and SpI.

### BRIEF DESCRIPTION OF THE FIGURES

These and other features of the invention will become more apparent from the following description in which reference is made to the appended drawings wherein:

FIG. 1 Illustrates a NF-κB electrophoretic mobility shift assay using nuclear extracts from brains. Control (lane 1), sham (lane 2), myocardial infarction (lane 3), Compound 1 (lane 4) and Compound 2 (lane 5).

FIG. 2 Provides a densitometric analysis of NF-κB EMSA bands.

FIG. 3 Illustrates a Spl electrophoretic mobility shift assay using nuclear extracts from brains. Control (lane 1), sham (lane 2), myocardial infarction (lane 3), Compound 1 (lane 4) and compound 2 (lane 5).

FIG. 4 Provides a densitometric analysis of Spl EMSA bands.

### DETAILED DESCRIPTION OF THE INVENTION

The present invention is directed to a method of treating stress by administering an effective amount of one or more zinc ionophores to a subject in need thereof. By “zinc ionophore” is meant a therapeutic compound complexed with zinc ions that is capable of carrying zinc ions across cell membranes. In accordance with the present invention “treating” includes preventing, blocking, inhibiting, attenuating, protecting against, modulating, reversing the effects of and reducing the occurrence of e.g., the harmful effects of stress. By “stress” is meant the broad range of alterations to normal homeostasis, for example, the complex hormonal and/or steroidal changes in a mammal, including a human that lead to changes in gene expression as exemplified by alterations in the activity of a number of transcription factors, including but not limited to NF-kB and Spl. Such hormonal and/or steroidal changes can result from direct and indirect physical and mental factors, such as, but not limited to injuries, trauma, surgical procedures, depression, anxiety and repressed or underexpressed worry, for example.

According to the present invention, small concentrations of a zinc ionophore in the nanomolar and picomolar range, such as from about 10 pM to about 1 μM can reverse the effects of stress. In another embodiment small concentrations of a zinc ionophore in the nanomolar and picomolar range, such as from about 10 pM to about 1 μM can regulate gene expression by modulating the activity of transcription factors in the various organ systems, including but not limited to the brain and heart of mammals, including humans. Transcription factors which may be modulated in accordance with the present invention include, but are not limited to NF-kB, AP-1 and Spl.

Thus, according to the present invention the concentration of zinc ionophore used to treat stress ranges from about 0.005 μg zinc ionophore per kg of body weight to about 5 mg zinc ionophore per kg of body weight (i.e. about 600 pM zinc ionophore to about 15 μM zinc ionophore). In a further embodiment of the present invention the concentration of zinc ionophore used to treat stress ranges from about 1.0 μg zinc ionophore per kg of body weight to about 800 μg zinc ionophore per kg of body weight. Preferably the concentration of zinc ionophore used to treat stress ranges from about 0.2 μg zinc ionophore per kg of body weight to about 600 μg zinc ionophore per kg of body weight.

In a further embodiment of the present invention the concentration of zinc ionophore used to treat stress is about 0.9 mg/kg body weight, or about 0.18 mg zinc/kg body weight.

According to the present invention, any compound capable of binding zinc with moderate affinity and having sufficient lipophilic properties to penetrate cell membranes is capable of effecting the protection demonstrated in the present invention with e.g., zinc-pyrithione. Zinc-pyrithione (zinc pyridinethione, C₁₀H₈N₂O₂S₂Zn, MW 317.75, commercially available from Sigma) is the active ingredient in the antidandruff shampoo Head & Shoulders® (U.S. Pat. Nos. 3,236,733, and 3,281,366, both 1966), as well as a number of other topical skin treatment formulations. It is a fungicide and bactericide at high concentrations. It is highly lipophilic and therefore penetrates membranes easily. This permits zinc pyrithione to transport zinc across cell membranes, thereby conferring on this compound (i.e. zinc pyrithione) the properties of a zinc ionophore.

In addition to zinc-pyrithione, another group of zinc ionophores, the dithiocarbamates, can treat stress in accordance with the present invention. The following are examples of compounds which have been shown in accordance with the present invention to possess zinc-ionophore properties: zinc pyrithione, the heterocyclic amines including, for example, 5,7-Diiodo-8-hydroxyquinoline, and 8-Hydroxyquinoline; the dithiocarbamates including, for example, pyrrolidine dithiocarbamate and diethyldithiocarbamate, disulfiram and dimethyldithiocarbamate; and Vitamins including, but not limited to, Vitamin E and Vitamin A. Properties associated with zinc ionophores include, but are not limited to, an ability to alter cytosolic PKC-content and an ability to alter the nuclear activity of transcription factors NF-kB, AP-1 and Spl. According to the present invention zinc-pyrithione was shown to operate at the cell signalling level, as demonstrated by its ability to alter cytosolic PKC-content. Further, according to the present invention, zinc-pyrithione was shown to operate at the transcriptional level, as demonstrated by its ability to alter the nuclear activity of transcription factors NF-kB, AP-1 and Spl. Still further, according to the present invention zinc-pyrithione was shown to upregulate cytoprotective proteins, for example HSP70.

In accordance with the present invention the zinc ionophores protect against the deleterious effects of stress. For example, both sham surgery and coronary occlusion had a similar effect on NF-kB and Spl transcription factor activity in the brain, suggesting that it was the stress associated with the surgical protocol, rather than the myocardial infarct itself, that caused this effect. In one embodiment of the present invention zinc ionophores displayed a strong ability to reverse the effects of surgical stress, or other forms of stress, in patients, including humans.

In accordance with the present invention, alterations in the activity of a number of transcription factors, including but not limited to NF-kB and Spl can modulate the neuronal response to the stress. Stress is also understood, in accordance with the present invention, to mean a mentally or emotionally disruptive or upsetting condition occurring in response to adverse external influences such as a surgical procedure or an injury which is capable of affecting physical health, usually characterized by increased heart rate, a rise in blood pressure, muscular tension, pain, irritability, and depression. The zinc ionophores of the present invention can be used to modulate and/or reverse the effects of stress generally, and in particular, surgical stress on the activity of transcription factors, including but not limited to NF-kB and Spl. In accordance with the present invention, zinc ionophores can modulate the effects of the surgical stress, or other forms of stress, in patients, including humans. The zinc ionophores of the present invention can be used at concentrations ranging from0.005 μg zinc ionophore per kg of body weight to about 5 mg zinc ionophore per kg of body weight (i.e. about 600 pM zinc ionophore to about 15 μM zinc ionophore) to treat stress and especially surgical stress.

In a further embodiment of the present invention the concentration of zinc ionophore used in a method to modulate and/or reverse the effects of surgical stress, or other forms of stress, in mammalian patients, ranges from about 1.0 μg zinc ionophore per kg of body weight to about 800 μg zinc ionophore per kg of body weight. In still another embodiment, the concentration of zinc ionophore used to modulate and/or reverse the effects of surgical stress, or other forms of stress, in mammalian patients ranges from about 0.2 μg zinc ionophore per kg of body weight to about 600 μg zinc ionophore per kg of body weight.

In use, at least one zinc ionophore, according to the present invention is administered in a pharmaceutically effective amount to a subject in need thereof in a pharmaceutical carrier by intravenous, intramuscular, subcutaneous, or intracerebroventricular injection or by oral administration or topical application. In accordance with the present invention, one zinc ionophore may be administered, preferably by the intravenous injection route, alone or in conjunction with a second, different zinc ionophore. By “in conjunction with” is meant together, substantially simultaneously or sequentially. In one embodiment, the zinc ionophores of the present invention, are administered acutely, such as, for example, substantially immediately following an injury that results in stress, such as surgery. The zinc ionophores may therefore be administered for a short course of treatment, such as for about 1 day to about 1 week. In another embodiment, the zinc ionophores of the present invention may be administered over a longer period of time to ameliorate chronic stress, such as, for example, for about one week to several months depending upon the condition to be treated.

By “pharmaceutically effective amount” as used herein is meant an amount of zinc ionophore, e.g., zinc-pyrithione, high enough to significantly positively modify the condition to be treated but low enough to avoid serious side effects (at a reasonable benefit/risk ratio), within the scope of sound medical judgment. A pharmaceutically effective amount of zinc ionophore will vary with the particular goal to be achieved, the age and physical condition of the patient being treated, the severity of the underlying disease, the duration of treatment, the nature of concurrent therapy and the specific zinc ionophore employed. For example, a therapeutically effective amount of a zinc ionophore administered to a child or a neonate will be reduced proportionately in accordance with sound medical judgment. The effective amount of zinc ionophore will thus be the minimum amount which will provide the desired anti-stress effect.

A decided practical advantage of the present invention is that the zinc ionophore, e.g. zinc-pyrithione, may be administered in a convenient manner such as by the, intravenous, intramuscular, subcutaneous, oral or intra-cerebroventricular injection routes or by topical application, such as in eye drops or eye mist compositions. Depending on the route of administration, the active ingredients which comprise zinc ionophores may be required to be coated in a material to protect the zinc ionophores from the action of enzymes, acids and other natural conditions which may inactivate the zinc ionophores. In order to administer zinc ionophores by other than parenteral administration, the ionophores can be coated by, or administered with, a material to prevent inactivation. For example, the zinc ionophores of the present invention may be co-administered with enzyme inhibitors or in liposomes. Enzyme inhibitors include pancreatic trypsin inhibitor, and trasylol. Liposomes include water-in-oil-in-water P40 emulsions as well as conventional and specifically designed liposomes.

The zinc ionophores may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the zinc ionophore in the required amount in the appropriate solvent with various of the other ingredients enumerated above, as required, followed by filtered sterilization. Generally, dispersions are prepared by incorporating the various sterilized zinc ionophores into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the zinc ionophores may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains a zinc ionophore concentration sufficient to treat or block apoptosis or stress in a patient.

The tablets, troches, pills, capsules, and the like, may contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil or wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules or zinc ionophore in suspension may be coated with shellac, sugar or both.

A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the zinc ionophore may be incorporated into sustained-release preparations and formulations.

By “pharmaceutically-acceptable carrier” as used herein is meant one or more compatible solid or liquid filler diluents or encapsulating substances. By “compatible” as used herein is meant that the components of the composition are capable of being comingled without interacting in a manner which would substantially decrease the pharmaceutical efficacy of the total composition under ordinary use situations.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

Accordingly, in a preferred form of treating stress the subject is administered a therapeutically effective amount of at least one zinc ionophore and a pharmaceutically acceptable carrier in accordance with the present invention. A preferred subject is a human. A preferred zinc ionophore is zinc pyrithione. Another preferred zinc ionophore is zinc diethyldithiocarbamate.

Various modifications may be made without departing from the invention. The disclosure is to be construed as exemplary, rather than limiting, and such changes within the principles of the invention as are obvious to one skilled in the art are intended to be included within the scope of the claims.

The present invention will now be demonstrated using specific examples that are not to be construed as limiting.

### EXAMPLE 1

### Screening for ionophores

Cell Cultures

Human umbilical vein endothelial cells (HUVEC) were purchased from Clonetics (San Diego, Calif.) and passages DeKeyser F. G. et al. (2000) Neuroimmunomodulation 7:182-188; Revilla V. et al. Brain Res. Bull 49:413-418; Troullos E. et al. (1997) Clin. Pharmacol. Ther. 62:74-81 were used for these studies. Cells were cultured on flame-sterilzed glass coverslips in Endothelial Basal Medium (Clonetics) supplemented with 10 ng/ml human recombinant epidermal growth factor, 1.0 ug/ml hydrocortisone, 50 ug/ml gentamicin, 50ng/ml amphotetericin B, 12 ug/ml bovine brain extract and 2% v/v fetal bovine serum (all from Clonetics), in a humidified chamber at 37° C. and 5% CO₂. To maintain cell populations, proliferating HUVEC were passaged at 80-90% confluency.

Cardiac myocytes were isolated from the ventricular septum of adult rabbit hearts, following collagenase digestion, in a manner similar to that described previously (Turan, B. et al., (1997) Am. J. Physiol. 272:H2095-H2106). The modification consisted of introducing low concentrations of CaCl₂ during the perfusion with collagenase and the dispersion of the myocytes. Hearts were perfused for about 2 min by gravity under a hydrostatic pressure of 1 m, with a nominally Ca²⁺-free solution containing (in mM): NaCl, 145; KCl, 5; MgSO₄, 1.2; Na₂HPO₄, 1.8; HEPES, 5; glucose, 10; pH adjusted to 7.4 with NaOH. Forty ml of this perfusate were then supplemented with collagenase (1 mg/ml) and perfusion was continued with recirculation. Within 2-3 min, this treatment resulted in a complete loss of ventricular pressure. The flow rate was then adjusted to 15 ml/min and 50 μM CaCl₂ was added to the collagenase solution. Perfusion with this solution was continued for another 15 to 18 min, followed by a 2 min washout of the enzyme with fresh perfusate containing 100 μM CaCl₂ and no collagenase. The hearts were then removed from the apparatus and the ventricular septum isolated and minced. Dissociation of the cells was obtained by gentle agitation of the minced tissue in 50 ml of the same perfusing solution. Following filtration through a 200 μm nylon mesh, the cells were allowed to settle and the supernatant was replaced with a solution containing 2 mM CaCl₂. Cells were kept at 37° C. in this pre-oxygenated solution and were studied within 8 hours after isolation, cellular viability was ensured by regularly replacing the incubation solution.

Primary cultures of mouse cerebellar granule neurons were obtained from dissociated cerebella of postnatal day 8 or 9 mice according to the following protocol (Cregan et al., (1999) J. Neurosci. 19:7860-7869, incorporated herein by reference). Brains were removed and placed into separate dishes containing solution A (124 mM NaCl, 5.37 mM KCl, 1 mM NaH2 P04, 1.2 mM MgSO4, 14.5 mM D-(1)-glucose, 25 mM HEPES, 3 mg/ml BSA, pH 7.4) in which the cerebella were dissected, meninges removed, and tissue sliced into small pieces. The tissue was briefly centrifuged and transferred to solution A containing 0.25 mg/ml trypsin, then incubated at 37° C. for 18 min. After the addition of 0.082 mg/ml trypsin inhibitor (Boehringer Mannheim, Indianapolis, Ind.) and 0.25 mg/ml DNase I (Boehringer Mannheim), the tissue was incubated at 25° C. for 2 min. After a brief centrifugation, the resulting pellet was gently titrated in solution A yielding suspension that was further incubated for 10 min at 25° C. in solution A containing 2.7 mM MgSO4 and 0.03 mM CaCl2. After a final centrifugation the pellet was resuspended in EMEM media (Sigma, St. Louis, Mo.) containing 10% dialyzed FBS (Sigma), 25 mM KCl, 2 mM glutamine (Life Technologies BRL, Gaithersburg, Md.), 25 mM glucose, and 0.1 mg/ml gentamycin (Sigma) and filtered through a cell strainer (size 70 μm; Falcon). Cells were plated on glass coverslips coated with poly-D-lysine (Sigma) in Nunc fourwell dishes at a density of 1.5×10⁶ cells per milliliter of medium. Cytosine-arabinoside (10 μM; Sigma) was added 24 hr after plating.

Test compounds

Several test compounds with potential zinc-ionophore activity were screened for their ability to transport zinc into selected target cells. In order to ascertain that the transported ion was indeed Zn²⁺, and not some other divalent cation contaminant, the test compounds were first complexed with zinc. In addition to the zinc-complexed ionophores (holo-ionophores), the zinc-free forms of these compounds (apo-ionophores) were also tested for the purpose of comparison. Whenever possible, purified holo-ionophores were purchased commercially (e.g. zinc-diethyldithiocarbamate, Sigma-Aldrich). However, in most cases only the apo-ionophores were available commercially. The holo-ionophores were therefore prepared in our laboratory. Since zinc ionophores (e.g. pyrithione, diethyldithiocarbamate, 8-hydroxyquinoline) complex with zinc in a 2:1 molar ratio (ionophore:zinc), stock solutions (generally 15.7 mM) of holo-ionophores were prepared by combining the apo-ionophore with ZnCl₂ in a 2:1 molar ratio either in water or DMSO, depending on the solubility of the reactants, and incubating at room temperature for 15 min. The holo-ionophores were then stored at −20° C. Immediately prior to screening, the stock solutions of these test compounds were thawed and diluted in the superfusion buffer to give a final concentration of 1 μM of the holo-ionophore. When testing the apo-ionophores, an equivalent molar concentration of the ionophore in the superfusion buffer (2 μM) was used.

Ionophore screening

Screening of the test compounds was performed with cultured HUVEC, isolated cardiac myocytes, and cultured cerebellar neurons following an approach described previously (Turan et al., (1997) Am. J. Physiol. 272:H2095-H2106). Immediately prior to screening, the cells were loaded with Fura-2, a zinc and calciumsensitive indicator, by incubating the cells for 30 min in medium containing 4 μM Fura-2-am (Molecular Probes). Glass coverslips bearing HUVEC or cerebellar cells were placed directly in a superfusion chamber on the stage of an epifluorescence inverted microscope (Nikon Diaphot-DM). With isolated myocytes, an aliquot of Fura-2 loaded cell suspension was placed in the superfusion chamber and the cells were allowed to adhere to the glass bottom of the chamber before superfusion was started. The microscope field of view was adjusted to include one or more individual cells. To establish baseline fluorescence, the cells were first superfused for a few minutes with a superfusing solution containing the following (in mM): NaCl, 140; KCl, 5; MgCl₂, 1; CaCl₂, 2, HEPES, 5; glucose, 10; pH adjusted to 7.4 with NaOH. The flow rate was maintained at approximately 3 ml/min and the temperature at 37° C. The cells were then superfused with superfusion buffer containing a test compound and the fluorescence at 505 nm was recorded in response to excitation at 340 nm and 380 nm. The slope of the fluorescence intensity ratio in response to excitation at 340 and 380 nm was used to determine ionophore activity. In each test, the membrane-permeant heavy metal chelator N,N,N′,N′,-tetrakis(2-pyridylmethyl)ethylenediamine (TPEN, 30 μM) was added to the superfusate at the end of the run. Since TPEN does not chelate Ca²⁺, loss of fluorescence in response to TPEN addition confirmed that the fluorescence was attributable to zinc. In cases where test holo-ionophores did not demonstrate zinc-ionophore activity, the validity of the negative observations was confirmed by adding zinc-pyrithione (1 μM) to the superfusing solution at the end of the test. An increase in fluorescence in response to the added zinc-pyrithione confirmed that the cell being tested was viable and responsive.

Approximately 50 test compounds were screened for ionophore activity using this approach. Of those, three groups of compounds were found to be particularly active zinc ionophores: pyrithione, dithiocarbamates, and hydroxyquinolines (See Table 1). Several compounds which do not belong to these groups also showed ionophore activity but at a lower level. The ionophore activity of pyrithione appeared to be comparable in all three cell types tested, as were the activities of diethyldithiocarbamate and 5,7-diiodo-8-hydroxyquinoline.

TABLE 1
EXAMPLE OF ZINC IONOPHORES

| | |
|---|---|
| ✓✓✓ | - excellent |
| ✓✓ | - very good |
| ✓ | - good |

ZINC-PYRITHIONE ✓✓✓
ZINC-DITHIOCARBAMATES
Pyrrolidinedithiocarbamate ✓✓
diethyldithiocarbamate ✓✓
Disulfiram ✓✓
dimethyldithiocarbamate ✓✓
ZINC-HETEROCYCLIC AMINES
8-Hydroxyquinoline, ✓
5,7-Diiodo-8-hydroxyquinoline ✓✓
ZINC-NSAID
Indomethacin ✓
ZINC-VITAMINS
Vitamin A (all-trans-retinol) ✓
Vitamin E (alpha-tocopherol)

### EXAMPLE 2

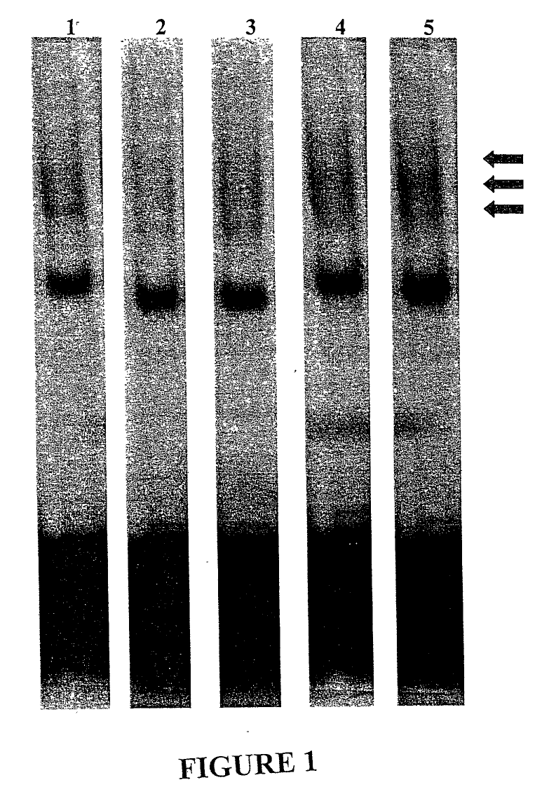

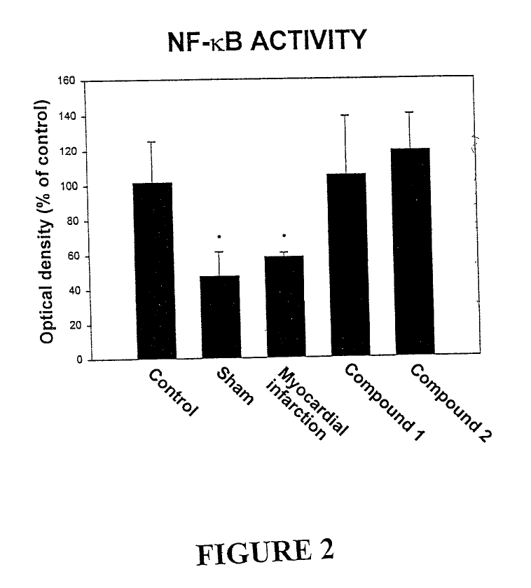

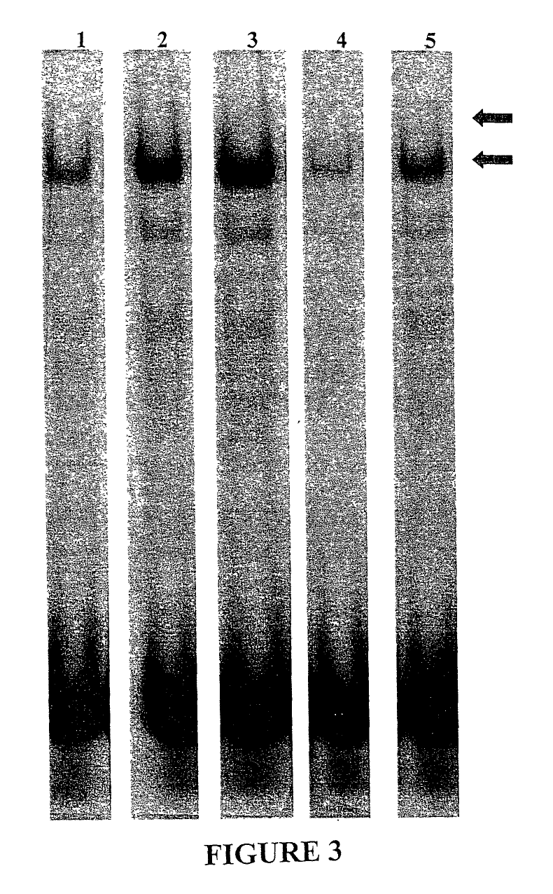

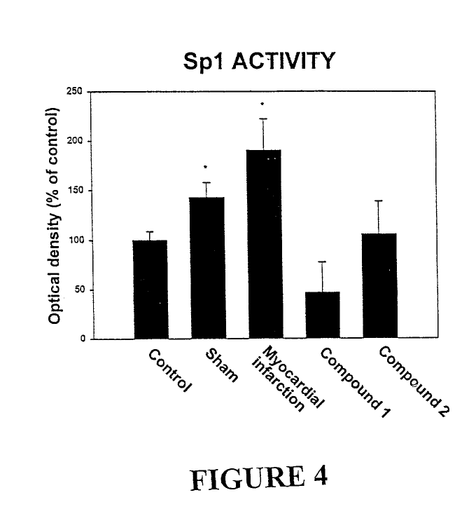

Sham surgery caused a statistically significant decrease in NF-κB binding activity in the brain (FIGS. 1 and 2). Myocardial infarction also decreased NF-κB activity but this decrease was not significantly different from that observed with the sham (FIG. 2). Treatment with ZnP (Compound 1) or ZNDDC (Compound 2) restored normal NF-κB binding activity in rats subjected to the coronary occlusion (FIG. 2). In contrast, sham treatment caused an increase in Spl binding activity in the brain (FIGS. 3 and 4). The effects of coronary occlusion were not significantly different from sham (FIG. 4). Treatment of rats subjected to coronary occlusion with ZnP (Compound 1) lowered Spl binding activity to nearly 50% of control (FIG. 4), whereas treatment with ZNDDC (Compound 2) restored Spl binding activity to normal (FIG. 4).

Both sham surgery and coronary occlusion had a similar effect on NF-kB and Spl transcription factor activity in the brain, suggesting that it was the stress associated with the surgical protocol, rather than the myocardial infarct itself, that caused this effect. The zinc ionophores of the present invention displayed a strong ability to reverse the effects of the surgical stress, or other forms of stress, in patients, including humans.

Coronary artery ligation model Male Sprague-Dawley rats (250-300 g) were anesthetized, intubated and ventilated. The chest was opened and the coronary artery was ligated. After 45 min, the suture was released and reperfusion was continued for 4 h. Boluses (0.5 ml) of the zinc ionophores ZnP (1.2 μg/kg) and ZnDDC (8.3 ng/kg) were administered intravenously in 4% DMSO in saline at 0, 1, and 2 h postreperfusion. After 4 h of reperfusion the ligature was retied, and Evans blue dye was infused intravenously to demarcate the area at risk. The rats were then killed by exsanguination and the brain was collected for analysis. Sham-surgery rats were treated in an identical manner but without tying the ligature around the coronary artery.

Preparation of nuclear extracts from brain tissue

Brain samples were homogenized on ice using six slow strokes of a Teflon pestle homogenizer at 1000 rpm in 8 volumes of buffer containing 0.25 M sucrose, 10 mM HEPES, pH 7.6, 25 mM KCl, 1 mM EDTA, 10% glycerol, 0.15 mM spermine, and 0.5 mM spermidine. The homogenate was filtered through a 45 mm nylon sieve and layered over a 10 ml cushion of 2 M sucrose containing 10 mM HEPES, pH 7.6, 25 mM KCl, 1 mM EDTA, and 10% glycerol. The homogenate was centrifuged at 100,000 xg at 4⁰C for 1 h, the supernatant was discarded, and the pelleted nuclei were gently resuspended in 40 ml of a lysis buffer containing 20 mM HEPES, pH 7.9, 420 mM NaCl, 1.5 mM MgCl₂, 0.2 mM EDTA, 25% glycerol, 0.5 mM DTT, 0.5 mM PMSF, 0.5 mM spermidine, 0.15 mM spermine, and 5 mg/ml each of aprotinin, leupeptin and pepstatin. The suspension was incubated on ice for 45 min and centrifuged at 20,000 xg at 4⁰C for 10 min. The supernatant containing nuclear protein was collected and diluted 1:1 with a buffer containing 20 mM HEPES, pH 7.9, 50 mM KCl, 0.2 mM EDTA, 20% glycerol, 0.5 mM DTT, 0.5 mM PMSF, 0.5 mM spermidine, 0.15 mM spermine, and 5 mg/ml each of aprotinin, leupeptin and pepstatin. Protein concentrations were determined using the Bio Rad protein assay.

EMSA (electrophoretic mobility shift assays)

Double-stranded consensus oligonucleotides for NF-kB and Spl (Promega, Madison, Wis.) were 5 radiolabelled with g[³²P]ATP (Amersham, Arlington Heights, Ill.). Five mg of nuclear protein were first incubated for 10 min at room temperature with 5 mg poly-d[I-C] (Boehringer Manheim, Montreal, Quebec) in DNA binding buffer (20 mM HEPES, pH 7.9, 0.2 mM EDTA, 0.2 mM EGTA, 100 mM KCl, 5% glycerol, and 2 mM DTT). Labelled probe (0.2 ng) was then added and the reaction mix incubated for a an additional 20 min in a final volume of 20 ml. The reaction mixture was subjected to electrophoresis on 5% polyacrylamide gel, and the dried gel was exposed to X-ray film. The intensity of the bands was quantitated with a densitometer and commercially available software (Molecular Analyst, Bio-Rad Laboratories, Hercules, Calif.).

What is claimed is:

1. A method of treating stress comprising administering to a patient in need thereof a pharmaceutically effective amount of a zinc ionophore and a pharmaceutically acceptable carrier.

2. The method of claim 1, wherein the zinc ionophore comprises zinc-pyrithione, zinc-heterocyclic amines, zinc-dithiocarbamates and zinc-vitamins.

3. The method of claim 2, wherein the zinc ionophore is zinc pyrithione.

4. The method of claim 2, wherein said zinc-heterocyclic amine comprises zinc-5,7-Diiodo-8-hydroxyquinoline and zinc-8-Hydroxyquinoline.

5. The method of claim 2, wherein said zinc-dithiocarbamate comprises zinc-pyrrolidine dithiocarbamate, zinc-diethyldithiocarbamate, zinc-disulfiram and zinc-dimethyldithiocarbamate.

6. The method of claim 2, wherein said zinc-vitamin is selected from the group consisting of Vitamin E and Vitamin A.

7. The method of claim 1, wherein the effective amount of a zinc ionophore ranges from about 0.005 μg per kg of body weight to about 5.0 mg per kg of body weight.

8. The method of claim 1, wherein the zinc ionophore is administered intravenously, intramuscularly, subcutaneously, intracerebroventricularly, orally or topically.

9. A method of reversing the effects of surgical stress comprising administering to a patient in need thereof a pharmaceutically effective amount of a zinc ionophore and a pharmaceutically acceptable carrier.

10. The method of claim 9, wherein the zinc ionophore comprises zinc- pyrithione, zinc-heterocyclic amines, zinc-dithiocarbamates and zinc-Vitamins.

11. The method of claim 10, wherein the zinc ionophore is zinc pyrithione.

12. The method of claim 10, wherein said zinc-heterocyclic amine comprises zinc-5,7-Diiodo-8-hydroxyquinoline and zinc-8-Hydroxyquinoline.

13. The method of claim 10, wherein said zinc-dithiocarbamate comprises zinc-pyrrolidine dithiocarbamate, zinc-diethyldithiocarbamate, zinc-disulfiram and zinc-dimethyldithiocarbamate.

14. The method of claim 10, wherein said zinc-vitamin is selected from the group consisting of Vitamin E and Vitamin A.

15. The method of claim 9, wherein the effective amount of a zinc ionophore ranges from about 0.005 μg per kg of body weight to about 5.0 mg per kg of body weight.

16. The method of claim 9, wherein the zinc ionophore is administered intravenously, intramuscularly, subcutaneously, intracerebroventricularly, orally or topically.

17. A method of regulating gene expression comprising modulating the activity of transcription factors by administering to a patient in need thereof a pharmaceutically effective amount of a zinc ionophore and a pharmaceutically acceptable carrier.

18. The method of claim 17, wherein the zinc ionophore comprises zinc-pyrithione, zinc-heterocyclic amines, zinc-dithiocarbamates and zinc-Vitamins.

19. The method of claim 18, wherein the zinc ionophore is zinc pyrithione.

20. The method of claim 18, wherein said zinc-heterocyclic amine comprises zinc-5,7-Diiodo-8-hydroxyquinoline and zinc-8-Hydroxyquinoline.

21. The method of claim 18, wherein said zinc-dithiocarbamate comprises zinc-pyrrolidine dithiocarbamate, zinc-diethyldithiocarbamate, zinc-disulfiram and zinc-dimethyldithiocarbamate.

22. The method of claim 18, wherein said zinc-vitamin is selected from the group consisting of Vitamin E and Vitamin A.

23. The method of claim 17, wherein the effective amount of a zinc ionophore ranges from about 0.005 μg per kg of body weight to about 5.0 mg per kg of body weight.

24. The method of claim 17, wherein the zinc ionophore is administered intravenously, intramuscularly, subcutaneously, intracerebroventricularly, orally or topically.

25. A method of protecting against the effects of stress comprising administering to a patient in need thereof a pharmaceutically effective amount of a zinc ionophore and a pharmaceutically acceptable carrier.

26. The method of claim 25, wherein the zinc ionophore comprises zinc-pyrithione, zinc-heterocyclic amines, zinc-dithiocarbamates and zinc-Vitamins.

27. The method of claim 26, wherein the zinc ionophore is zinc pyrithione.

28. The method of claim 26, wherein said zinc-heterocyclic amine comprises zinc-5,7-Diiodo-8-hydroxyquinoline and zinc-8-Hydroxyquinoline.

29. The method of claim 26, wherein said zinc-dithiocarbamate comprises zinc-pyrrolidine dithiocarbamate, zinc-diethyldithiocarbamate, zinc-disulfiram and zinc-dimethyldithiocarbamate.

30. The method of claim 26, wherein said zinc-vitamin is selected from the group consisting of Vitamin E and Vitamin A.

31. The method of claim 25, wherein the effective amount of a zinc ionophore ranges from about 0.005 μg per kg of body weight to about 5.0 mg per kg of body weight.

32. The method of claim 25, wherein the zinc ionophore is administered intravenously, intramuscularly, subcutaneously, intracerebroventricularly, orally or topically.
